(19) United States
(12) Patent Application Publication
(10) Pub. No.: US 2014/0031673 A1
(43) Pub. Date: Jan. 30, 2014

(54) ULTRASONIC DIAGNOSTIC APPARATUS AND CONTROL PROGRAM THEREOF

(71) Applicant: GE Medical Systems Global Technology Company, LLC, Waukesha, WI (US)

(72) Inventors: Shinichi Amemiya, Tokyo (JP); Mitsuhiro Nozaki, Tokyo (JP)

(73) Assignee: GE Medical Systems Global Technology Company, LLC, Waukesha, WI (US)

(21) Appl. No.: 13/950,872

(22) Filed: Jul. 25, 2013

(30) Foreign Application Priority Data
Jul. 26, 2012 (JP) 2012-165285

(51) Int. Cl. A61B 8/08

(52) U.S. Cl. CPC A61B 8/0841
USPC 600/424; 600/459

(57) ABSTRACT

An ultrasonic diagnostic apparatus is provided. The ultrasonic diagnostic apparatus includes an ultrasonic probe having plural ultrasonic transducers arranged in an elevation direction, a beamformer configured to form an ultrasonic reception beam by performing delay addition to an echo signal received by each of the ultrasonic transducers, and configured to form plural ultrasonic reception beams, each ultrasonic reception beam having a different width in the elevation direction, wherein the beamformer is configured to form the plural ultrasonic reception beams for one transmission/reception surface by adjusting a delay time in the delay addition, and a display control unit configured to display a synthetic image formed based upon the plural ultrasonic reception beams.

### CROSS REFERENCE TO RELATED APPLICATIONS

This application claims the benefit of Japanese Patent Application No. 2012-165285 filed Jul. 26, 2012, which is hereby incorporated by reference in its entirety.

### BACKGROUND OF THE INVENTION

The present invention relates to an ultrasonic diagnostic apparatus that can change a width of an ultrasonic reception beam in an elevation direction, and its control program.

An ultrasonic diagnostic apparatus can display an ultrasonic image of a subject on a real-time basis. By utilizing this real-time property, the position of the biopsy needle is confirmed by a real-time ultrasonic image during the insertion of the biopsy needle into the subject.

However, when the biopsy needle is particularly thin, the biopsy needle is bent on the way, so that the biopsy needle might be outside the transmission/reception surface of the ultrasonic, i.e., outside the range of the ultrasonic beam formed by an ultrasonic probe. In this case, the biopsy needle that is outside the range of the ultrasonic beam cannot be confirmed in the ultrasonic image. In view of this, in an ultrasonic diagnostic apparatus described in JP-A No. 9(1997)-135498, an opening is adjusted to adjust a width of an ultrasonic beam in an elevation direction in order that the ultrasonic beam covers the biopsy needle. A synthetic image formed by synthesizing images formed by plural ultrasonic reception beams, each having a different width, is displayed.

However, in the ultrasonic diagnostic apparatus described in JP-A No. 9(1997)-135498, the opening width in the elevation direction is reduced in order to widen the width of the ultrasonic beam in the elevation direction. Therefore, the receiving sensitivity is deteriorated by the reduced width. Accordingly, deterioration in the quality of the synthetic image may occur.

Even if the opening is adjusted, the focal position in the depth direction is not changed, so that there is a limitation in adjusting the width of the ultrasonic beam. Therefore, the biopsy needle might not be covered. Accordingly, it may be desirable that ultrasonic beams having wide variety of widths can be set in order to surely cover the biopsy needle by the ultrasonic beam.

Accordingly, there has been a demand for an ultrasonic diagnostic apparatus that can variedly adjust the width of the ultrasonic beam in order that an image including a biopsy needle has satisfactory quality, and that the image more surely includes the biopsy needle.

### BRIEF DESCRIPTION OF THE INVENTION

In a first aspect, an ultrasonic diagnostic apparatus is provided. The ultrasonic diagnostic apparatus includes an ultrasonic probe having plural ultrasonic transducers in an elevation direction, a beamformer that forms an ultrasonic reception beam by performing delay addition to an echo signal received by each of the ultrasonic transducers, and that forms plural ultrasonic reception beams, each having a different width in the elevation direction, for one transmission/reception surface by adjusting a delay time in the delay addition, and a display control unit that displays a synthetic image formed based upon the plural ultrasonic reception beams.

In a second aspect, an ultrasonic diagnostic apparatus of the first aspect is provided, in which the beamformer sets a central frequency of a second ultrasonic transmission beam for acquiring a second ultrasonic reception beam to be lower than a central frequency of a first ultrasonic transmission beam for acquiring a first ultrasonic reception beam, transmits the second ultrasonic transmission beam in a direction generally orthogonal to a planned insertion path of the biopsy needle, and forms the second ultrasonic reception beam in the direction generally orthogonal to the planned insertion path.

In a third aspect, an ultrasonic diagnostic apparatus of the first aspect is provided, in which the beamformer sets a reception gain of the second ultrasonic reception beam to be higher in a region in which the biopsy needle can be inserted than in a region outside the region.

According to the first aspect, the width of the ultrasonic reception beam in the elevation direction is changed by adjusting the delay time without adjusting the opening, whereby the ultrasonic reception beam can be acquired without deteriorating the receiving sensitivity. Accordingly, quality of a synthetic image formed based upon plural ultrasonic reception beams, each having a different width in the elevation direction, can be more satisfactory than previously. By adjusting the delay time, the focal point of the ultrasonic reception beam can be more finely adjusted, whereby the width of the ultrasonic reception beam can more variedly be changed. Accordingly, the width of the ultrasonic reception beam can be adjusted so as to more surely cover the biopsy needle, whereby the biopsy needle can more surely be displayed in the synthetic image.

According to the second aspect, the central frequency of the second ultrasonic transmission beam is set to be lower than the central frequency of the first ultrasonic transmission beam, the second ultrasonic transmission beam is transmitted in the direction generally orthogonal to the insertion path of the biopsy needle, and the second ultrasonic reception beam in the direction generally orthogonal to the insertion path is formed. Accordingly, the biopsy needle can more clearly be displayed in the synthetic image.

According to the third aspect, the reception gain of the second ultrasonic reception beam is set to be higher in the region in which the biopsy needle can be inserted than in the region outside the region, whereby the biopsy needle can more clearly be displayed in the synthetic image. Since the reception gain is set higher in only some region, S/N of the synthetic image becomes more satisfactory, compared to the case where the reception gain is set high all over the region. Consequently, the synthetic image having satisfactory quality can be formed.

### BRIEF DESCRIPTION OF THE DRAWINGS

FIG. 1 is a block diagram illustrating one example of a schematic configuration of an ultrasonic diagnostic apparatus.

FIG. 2 is a plan view illustrating an ultrasonic transducer forming an ultrasonic probe.

FIG. 3 is a view illustrating an outer appearance of the ultrasonic probe.

FIG. 4 is a view for describing a position of a biopsy needle in an elevation direction.

FIG. 5 is a conceptual view illustrating a first ultrasonic transmission beam and a second ultrasonic transmission beam.

FIG. 6 is a view for describing the ultrasonic transducer used for transmitting the first ultrasonic transmission beam and the second ultrasonic transmission beam.

FIG. 7 is a view for describing one example of a first ultrasonic reception beam and a second ultrasonic reception beam.

FIG. 8 is a view for describing another example of the first ultrasonic reception beam and the second ultrasonic reception beam.

FIG. 9 is a view illustrating one example of a display unit on which a B-mode image is displayed.

FIG. 10 is a view for describing a transmission direction of the second ultrasonic transmission beam and a forming direction of the second ultrasonic reception beam in a first modification.

FIG. 11 is a view for illustrating a region where a gain of the second ultrasonic reception beam is set to be high in a second modification.

### DETAILED DESCRIPTION OF THE INVENTION

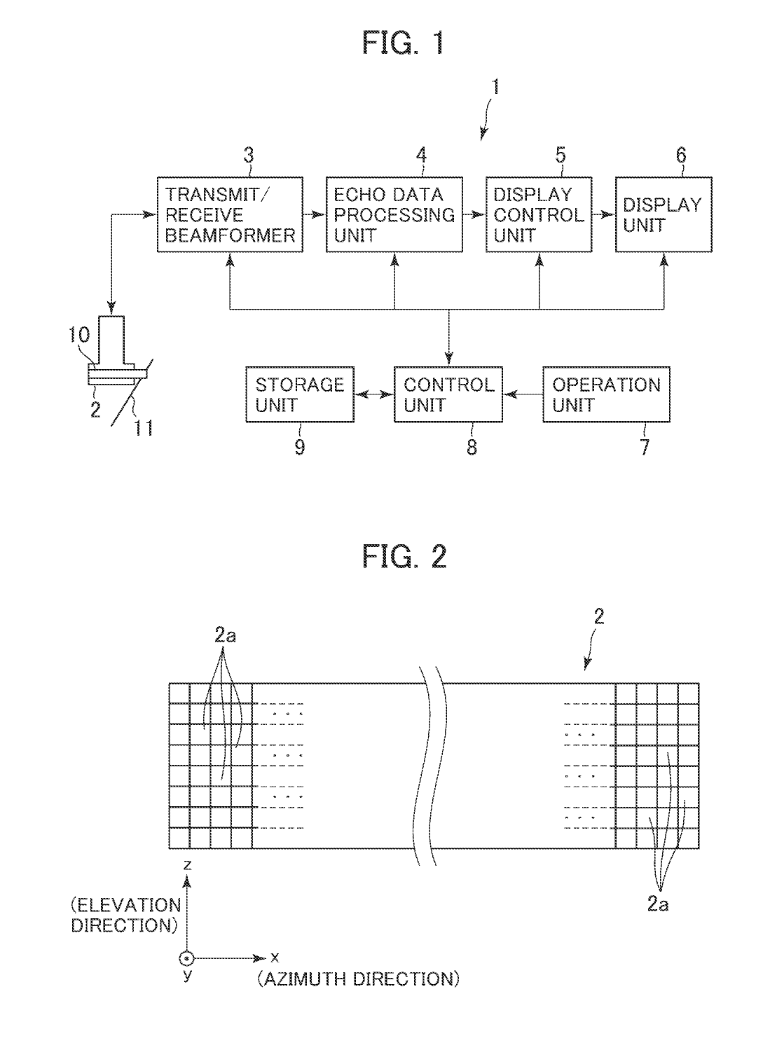

An exemplary embodiment will be described below with reference to FIGS. 1 to 9. An ultrasonic diagnostic apparatus 1 illustrated in FIG. 1 includes an ultrasonic probe 2, a transmit/receive beamformer 3, an echo data processing unit 4, a display control unit 5, a display unit 6, an operation unit 7, a control unit 8, and a storage unit 9.

As illustrated in FIG. 2, the ultrasonic probe 2 is configured to include plural ultrasonic transducers 2a arranged in an array. The ultrasonic probe 2 transmits an ultrasonic to a subject by use of the ultrasonic transducers, and receives its echo signal. Plural ultrasonic transducers 2a are arranged in an azimuth direction (x direction) and in an elevation direction (z direction).

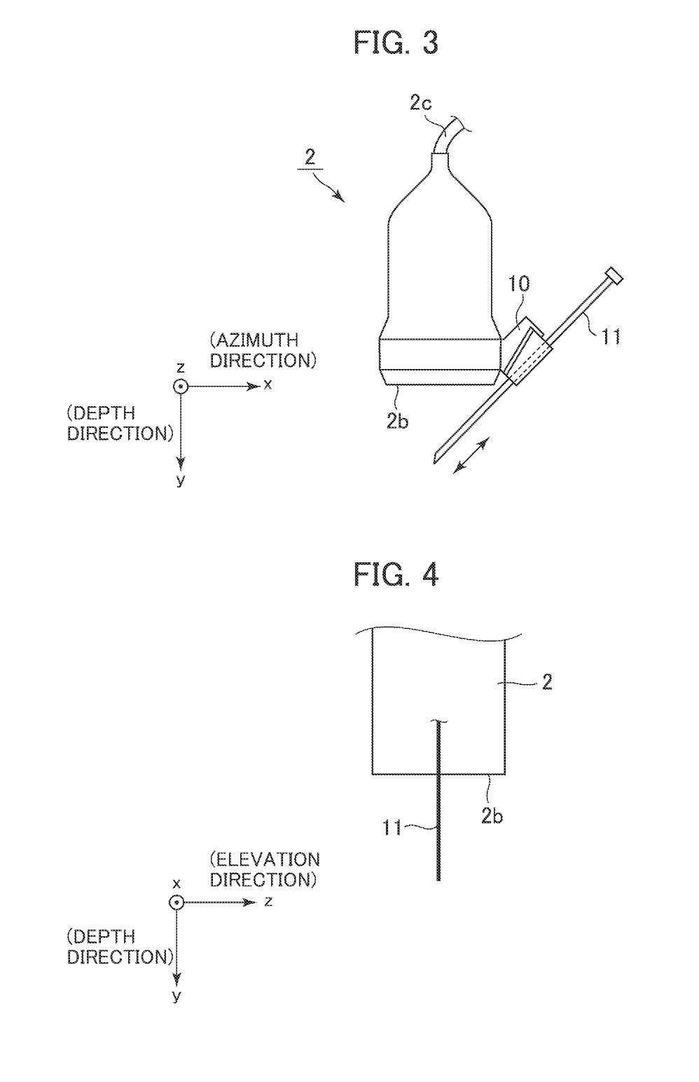

As illustrated in FIG. 3, an ultrasonic irradiation surface 2b for the ultrasonic is formed on a tip end of the ultrasonic probe 2. Although not particularly illustrated in FIG. 3, the irradiation surface 2b may be made of a convex acoustic lens. A probe cable 2c connected to a body (not illustrated) of the ultrasonic diagnostic apparatus 1 extends from the side, reverse to the tip end, of the ultrasonic probe 2.

A biopsy guide attachment 10 is detachably mounted near the ultrasonic irradiation surface 2b of the ultrasonic probe 2. A biopsy needle 11 can be mounted to the biopsy guide attachment 10 so as to be capable of moving forward and backward. The biopsy needle 11 attached to the biopsy guide attachment 10 is located on the end of the ultrasonic probe 2 in the azimuth direction in the state in which the biopsy guide attachment 10 is mounted to the ultrasonic probe 2. The biopsy needle 11 attached to the ultrasonic probe 2 via the biopsy guide attachment 10 can move forward and backward along the transmission/reception surface (scanning surface) of the ultrasonic.

As illustrated in FIG. 4, the biopsy needle 11 attached to the ultrasonic probe 2 via the biopsy guide attachment 10 is located almost on the center of the ultrasonic probe 2 in the elevation direction in the present embodiment. FIG. 4 briefly illustrates only the ultrasonic probe 2 and the biopsy needle 11, and does not illustrate the biopsy guide attachment 10.

The transmit/receive beamformer 3 feeds a signal for transmitting the ultrasonic from the ultrasonic probe 2 under a predetermined scanning condition to the ultrasonic probe 2 based upon a control signal from the control unit 8. In the exemplary embodiment, the transmit/receive beamformer 3 feeds the signal to the ultrasonic probe 2 in order that two types of transmission ultrasonic beams, which are a first transmission ultrasonic beam and a second transmission ultrasonic beam, each having a different beam shape, are formed as described later.

The transmit/receive beamformer 3 performs a signal process such as A/D conversion and delay adding process, and a signal process for amplifying the signal with a predetermined gain, to the echo signal received by the ultrasonic probe 2, thereby forming an ultrasonic reception beam. As described later, the transmit/receive beamformer 3 forms two types of reception ultrasonic beams, which are a first ultrasonic reception beam and a second ultrasonic reception beam, each having a different beam shape (beam-forming function). The detail will be described later. The transmit/receive beamformer 3 is one example of an embodiment of a beamformer.

The transmit/receive beamformer 3 outputs the echo data after the signal process to the echo data processing unit 4.

The echo data processing unit 4 performs a process for generating an ultrasonic image to the echo data outputted from the transmit/receive beamformer 3. For example, the echo data processing unit 4 performs a B-mode process including a logarithmic compression and an envelope detection, thereby generating a B-mode image. In the exemplary embodiment, the B-mode data is first B-mode data based upon the echo data forming the first ultrasonic reception beam, and second B-mode data based upon the echo data forming the second ultrasonic reception beam.

The display control unit 5 makes a scan conversion to the B-mode data by using a scan converter, thereby generating B-mode image data. The B-mode image data is first B-mode image data based upon the first B-mode data, and second B-mode image data based upon the second B-mode data. The B-mode data before the scan conversion is referred to as raw data.

The display control unit 5 synthesizes the first B-mode image data and the second B-mode image data to form synthetic image data. The display control unit 5 then displays a synthetic B-mode image based upon the synthetic image data onto the display unit 6 (display control function). The display control unit 5 is one example of an embodiment of a display control unit.

The display unit 6 is an LCD (Liquid Crystal Display) or CRT (Cathode Ray Tube). The operation unit 7 is configured to include a keyboard and a pointing device (not illustrated) that is used by an operator for inputting command or information.

The control unit 8 is a CPU (Central Processing Unit), and it reads a control program stored in the storage unit 9 to execute the functions, such as the beam-forming function and the display control function, in each unit of the ultrasonic diagnostic apparatus 1.

The storage unit 9 is, for example, HDD (Hard Disk Drive) or a semiconductor memory (memory).

The operation of the ultrasonic diagnostic apparatus 1 in the exemplary embodiment will be described. The transmit/receive beamformer 3 alternately transmits the first ultrasonic transmission beam and the second ultrasonic transmission beam to the same plane of a biological tissue of the subject from the ultrasonic probe 2 one frame by one frame.

The first ultrasonic transmission beam and the second ultrasonic transmission beam have different beam width. The transmit/receive beamformer 3 changes the beam width of the first ultrasonic transmission beam and the second ultrasonic transmission beam by changing the opening width of the ultrasonic probe 2 in the elevation direction.

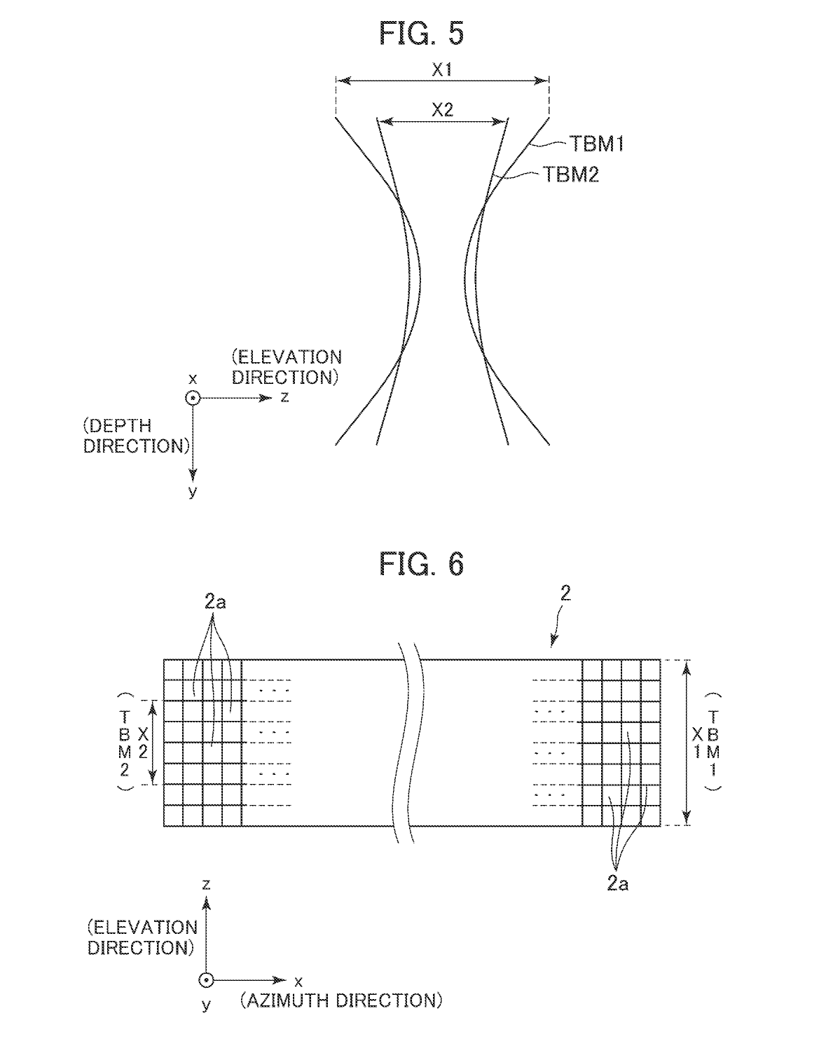

As illustrated in FIG. 5, an opening width X1 of the first ultrasonic transmission beam TBM1 is larger than an opening width X2 of the second ultrasonic transmission beam TBM2. The beam width of the first ultrasonic transmission beam TBM1 is smaller than the beam width of the second ultrasonic transmission beam TBM2.

When the number of ultrasonic transducers 2a in the ultrasonic probe 2 in the elevation direction is eight as illustrated in FIG. 6, for example, the first ultrasonic transmission beam TBM1 is formed (opening width X1) with all eight ultrasonic transducers 2b in the elevation direction transmitting the ultrasonic energy. On the other hand, the second ultrasonic transmission beam TBM2 is formed (opening width X2) by transmitting the ultrasonic energy using only four of eight ultrasonic transducers 2b in the elevation direction.

The first ultrasonic transmission beam TBM1 is a beam for an image of the biological tissue, while the second ultrasonic transmission beam TBM2 is a beam for the biopsy needle 11 inserted into the biological tissue. The beam width of the second ultrasonic transmission beam TBM2 is set so as to be capable of covering the biopsy needle 11 outside the range of the first ultrasonic transmission beam TBM1.

The transmit/receive beamformer 3 forms the first ultrasonic reception beam RBM1 to the first ultrasonic transmission beam TBM1 based upon the echo signal received by each of the ultrasonic transducers 2a. The transmit/receive beamformer 3 also forms the second ultrasonic reception beam RBM2 to the second ultrasonic transmission beam TBM2 based upon the echo signal received by each of the ultrasonic transducers 2a. Since the first ultrasonic transmission beam TBM1 and the second ultrasonic transmission beam TBM2 are alternately transmitted one frame by one frame, the first ultrasonic reception beam RBM1 and the second ultrasonic reception beam RBM2 are also alternately formed one frame by one frame.

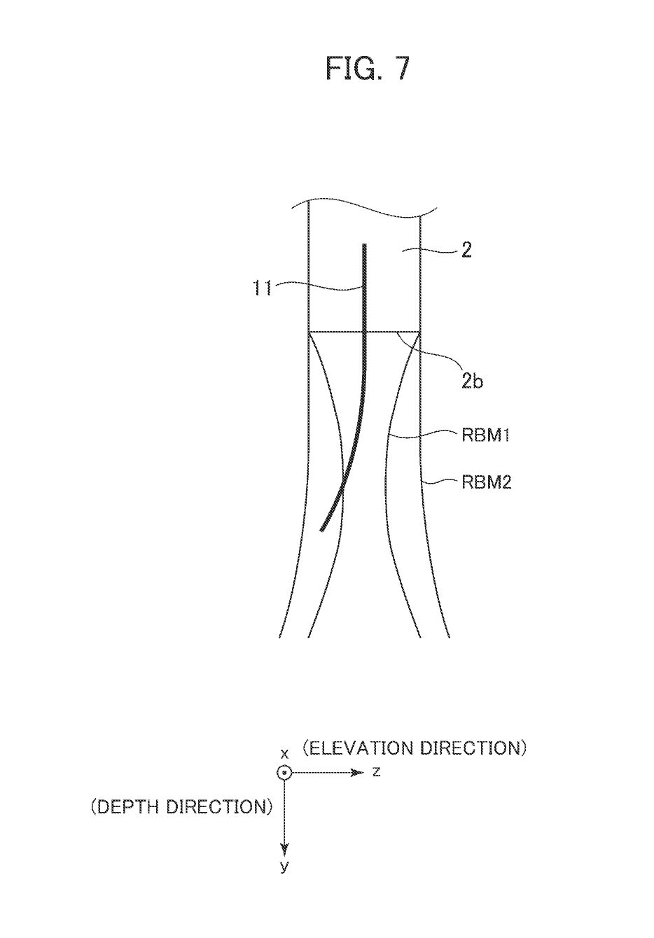

As illustrated in FIG. 7, the beam width of the first ultrasonic reception beam RBM1 and the beam width of the second ultrasonic reception beam RBM2 in the elevation direction are different from each other. The transmit/receive beamformer 3 adjusts a delay time when performing the delay adding process to the echo signal received by each of the ultrasonic transducers 2a, thereby changing the beam width of the first ultrasonic reception beam RBM1 and the beam width of the second ultrasonic reception beam RBM2. Therefore, the beam width of the first ultrasonic reception beam RBM1 and the beam width of the second ultrasonic reception beam RBM2 are changed without changing the opening width in the elevation direction. For example, all ultrasonic transducers 2a in the elevation direction receive the echo signal. As described above, the opening width is not changed for changing the beam width of the ultrasonic reception beam. Consequently, the receiving sensitivity of the echo signal can be maintained.

Although a scale is different between FIGS. 7 and 5, the opening width of the first ultrasonic reception beam RBM1 and the opening width of the second ultrasonic reception beam RBM2 in the elevation direction are the same as the opening width X1 of the transmission beam illustrated in FIG. 5.

The first ultrasonic reception beam RBM1 is a beam for an image of the biological tissue, while the second ultrasonic reception beam RBM2 is a beam for the biopsy needle 11 inserted into the biological tissue. The beam width of the first ultrasonic reception beam RBM1 is smaller than the beam width of the second ultrasonic reception beam RBM2 as illustrated in FIG. 7. The beam width of the first ultrasonic reception beam RBM1 is set such that the quality of the B-mode image generated based upon the first ultrasonic reception beam RBM1 is appropriate for observing the biological tissue.

On the other hand, the second ultrasonic reception beam RBM2 is the ultrasonic reception beam for the biopsy needle for forming the image of the biopsy needle 11, and it is set so as to be capable of covering the biopsy needle 11 outside the range of the first ultrasonic reception beam RBM1. The biopsy needle 11 inserted into the biological tissue might be bent on the way as illustrated in FIG. 7. The beam width of the second ultrasonic reception beam RBM2 is set to be larger than the beam width of the first ultrasonic reception beam RBM1 in order to be capable of covering the bent biopsy needle 11.

Plural beam widths of the second ultrasonic reception beam RBM2 may be set. For example, plural beam widths of the second ultrasonic reception beam RBM2 can be set according to the thickness of the biopsy needle 11. It may be set such that, the thinner the biopsy needle 11 becomes, the thicker the beam width of the second ultrasonic reception beam RBM2 becomes, since the thinner biopsy needle 11 may be easily bent. In this case, based upon the type (thickness) of the biopsy needle 11 inputted on the operation unit 7, the transmit/receive beamformer 3 may form the second ultrasonic reception beam RBM2 having the beam width according to the type of this biopsy needle 11.

Based upon the type (thickness) of the biopsy needle 11 inputted on the operation unit 7, the transmit/receive beamformer 3 may form the second ultrasonic transmission beam TBM2 having the beam width according to the type of this biopsy needle 11.

The transmit/receive beamformer 3 adjusts the beam width by adjusting the position of the focal point of the ultrasonic reception beam RBM2 in the depth direction (y direction) through the adjustment of the delay time. Therefore, when plural beam widths of the second ultrasonic reception beam RBM2 can be set, the delay time corresponding to each beam width of each second ultrasonic reception beam RBM2 is stored in the storage unit 9.

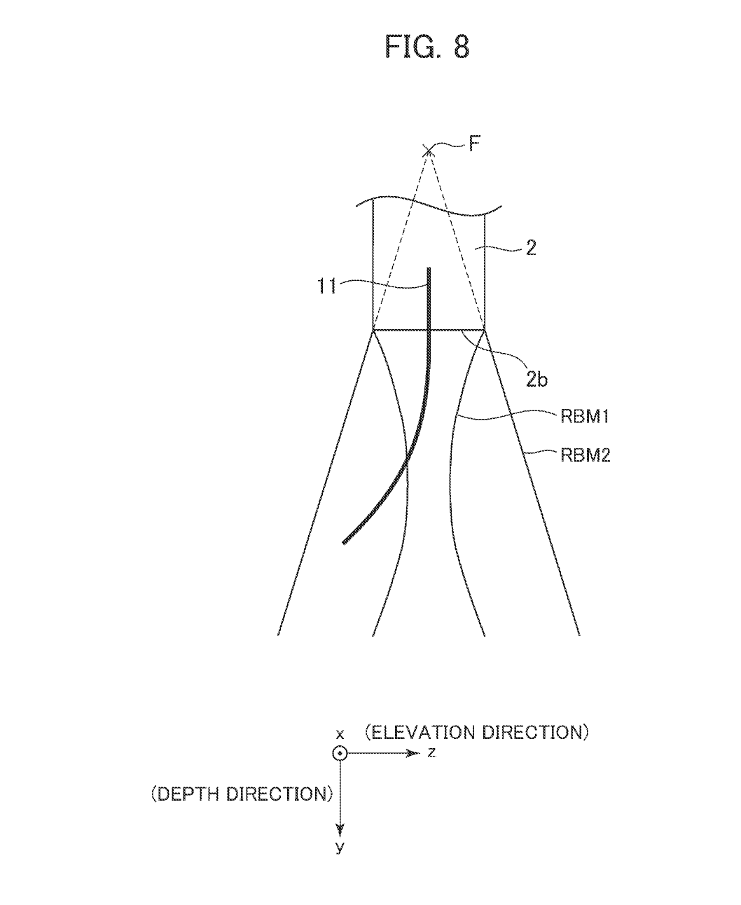

FIG. 7 illustrates the second ultrasonic reception beam RBM2 whose focal point (not illustrated) is set on the infinite distance. FIG. 8 illustrates the second ultrasonic reception beam RBM2 whose focal point F is set on the point of the ultrasonic probe 2 closer to the ultrasonic probe 2 (closer to the side opposite to the biological tissue) than to the irradiation surface 2b. By adjusting the delay time as described above, the focal position of the ultrasonic reception beam can be set on various positions in the depth direction, whereby the degree of freedom in setting the beam width can be increased. Accordingly, the appropriate beam width according to the bending way of the biopsy needle 11 can be set. Thus, the second ultrasonic reception beam RBM2 can more surely cover the biopsy needle 11.

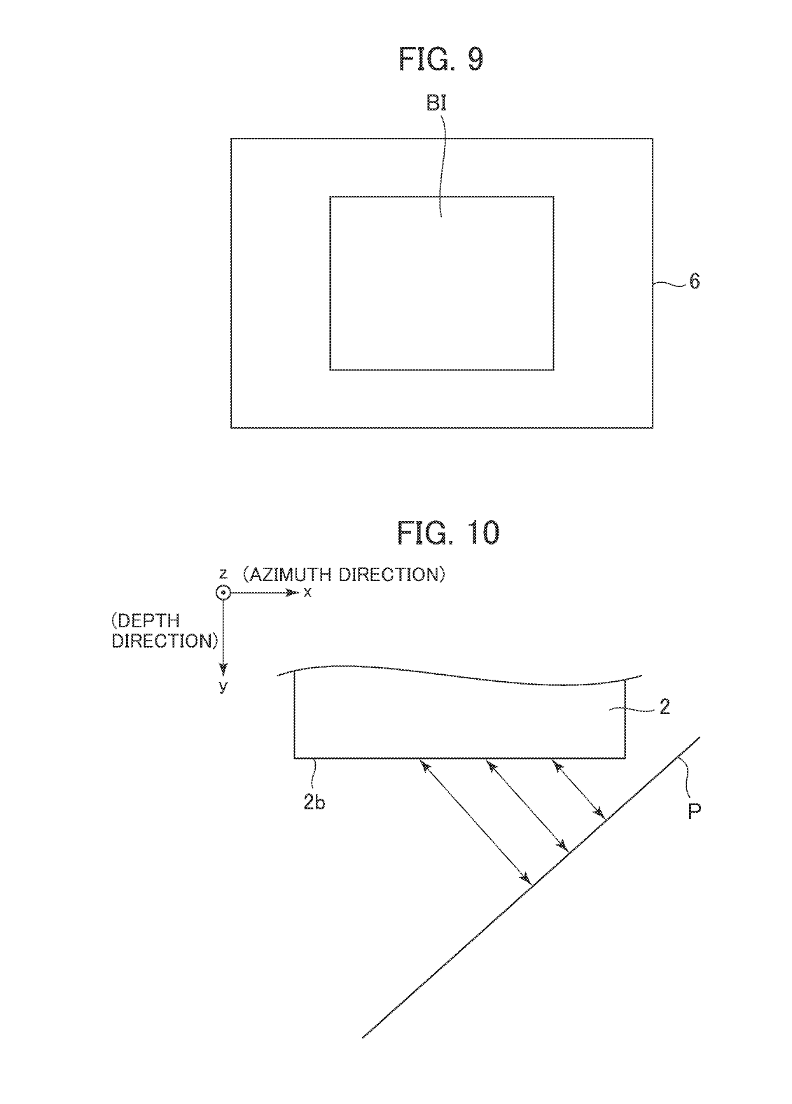

When the first ultrasonic reception beam RBM1 and the second ultrasonic reception beam RBM2 are formed by the transmit/receive beamformer 3, the echo data processing unit 4 generates the first B-mode data and the second B-mode data based upon the echo data forming the first ultrasonic reception beam RBM1 and the echo data forming the second ultrasonic reception beam RBM2. The display control unit 5 allows the display unit 6 to display a synthetic B-mode image BI based upon the synthetic image data, which is formed by synthesizing the first B-mode image data generated based upon the first B-mode data and the second B-mode image data generated based upon the second B-mode data, as illustrated in FIG. 9. The synthetic B-mode image BI is one example of an embodiment of the synthetic image.

Since the beam width of the first ultrasonic reception beam RBM1 is smaller than the beam width of the second ultrasonic reception beam RBM2, the image based upon the first B-mode image data keeps the resolution, so that it is appropriate for the observation of the biological tissue. On the contrary, since the beam width of the second ultrasonic reception beam RBM2 is larger than the beam width of the first ultrasonic reception beam RBM1 to cover the biopsy needle 11, the image based upon the second B-mode image data includes the biopsy needle 11. Therefore, the synthetic B-mode image BI formed by synthesizing the first B-mode image data and the second B-mode image data keeps the resolution of the biological tissue, and includes the biopsy needle 11.

In order to change the beam width of the ultrasonic reception beam, the delay time is adjusted without adjusting the opening width, whereby the receiving sensitivity of the echo signal can be maintained. Therefore, the quality of the synthetic B-mode image BI can be maintained, compared to the case where the beam width of the ultrasonic reception beam is changed by adjusting the opening width.

A first modification will next be described. In this modification, the transmit/receive beamformer 3 deflects and transmits the second ultrasonic transmission beam in the direction (direction of an arrow) generally orthogonal to a planned insertion path P of the biopsy needle 11 as illustrated in FIG. 10. The transmit/receive beamformer 3 forms the second ultrasonic reception beam that is deflected in the direction generally orthogonal to the planned insertion path P.

The planned insertion path P is set beforehand according to the type of the biopsy guide attachment 10. The planned insertion path P is a planned path through which the biopsy needle 11 is inserted when the biopsy needle 11 is inserted into the biological tissue straight along the guide of the biopsy guide attachment 10.

The transmit/receive beamformer 3 sets the central frequency of the second ultrasonic transmission beam to be lower than the central frequency of the first ultrasonic beam. Thus, the transmit/receive beamformer 3 can deflect and transmit the second ultrasonic transmission beam in the direction generally orthogonal to the planned insertion path P.

In this modification, the second ultrasonic transmission beam and the second ultrasonic reception beam in the direction orthogonal to the biopsy needle 11 inserted into the biological tissue can be formed. Accordingly, the biopsy needle 11 can be displayed more clearly on the synthetic B-mode image BI.

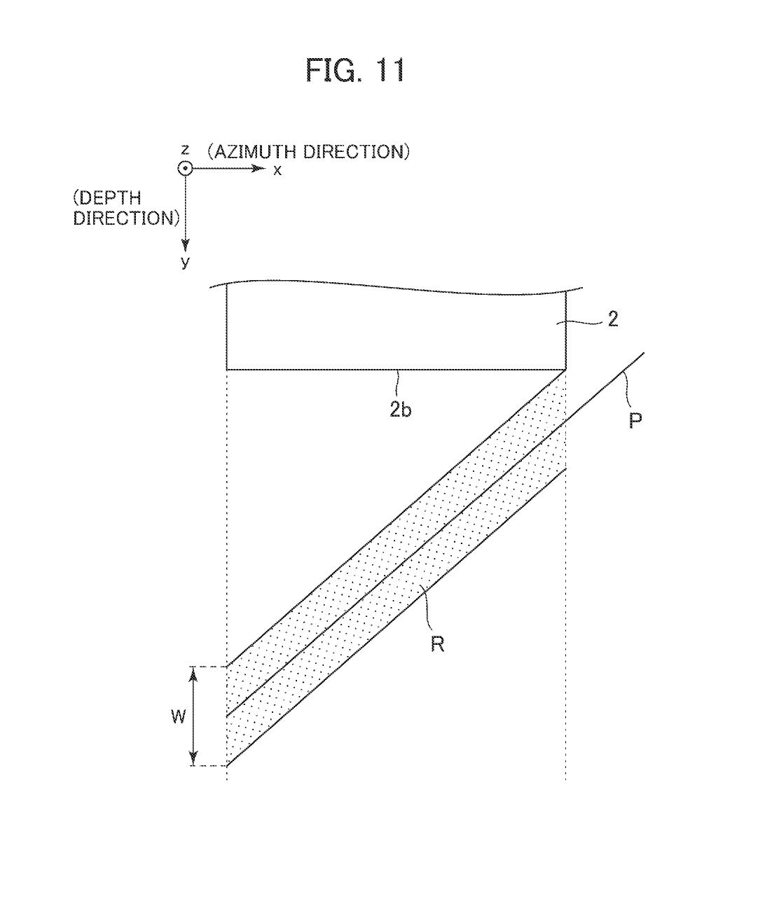

A second modification will next be described. The transmit/receive beamformer 3 may set the gain of the second ultrasonic reception beam (not illustrated in FIG. 11) to be higher in a region R in which the biopsy needle 11 can be inserted than in a region outside the region R. The region R is set with the planned insertion path P being defined as a reference. Specifically, the region R may be set to have a predetermined width W about the planned insertion path P as a centerline. This width W is set to have a size by which the biopsy needle 11 can be inserted.

Since the gain of the second ultrasonic reception beam is higher in the region R into which the biopsy needle 11 is inserted, the biopsy needle 11 can clearly be displayed in the synthetic B-mode image BI. Since the gain is set to be higher only in the region R, S/N in the synthetic B-mode image BI is enhanced, compared to the case where the gain is set to be high all over the region, whereby the synthetic B-mode image BI having satisfactory quality can be formed.

A third modification will next be described. The transmit/receive beamformer 3 may transmit the ultrasonic transmission beam of one frame for one transmission/reception surface, and may form the first ultrasonic reception beam and the second ultrasonic reception beam based upon the echo signal acquired by one-frame ultrasonic transmission beam.

While the disclosure has been described above using exemplary embodiments, various modifications are obviously possible without departing from the scope of the present invention. For example, a dynamic range may be different between the first B-mode image data and the second B-mode image data. A parameter in an edge enhance process or smoothing process may be different between the first B-mode image data and the second B-mode image data.

1. An ultrasonic diagnostic apparatus comprising:
an ultrasonic probe having plural ultrasonic transducers arranged in an elevation direction;
a beamformer configured to form an ultrasonic reception beam by performing delay addition to an echo signal received by each of the ultrasonic transducers, and configured to form plural ultrasonic reception beams, each ultrasonic reception beam having a different width in the elevation direction, wherein the beamformer is configured to form the plural ultrasonic reception beams for one transmission/reception surface by adjusting a delay time in the delay addition; and
a display control unit configured to display a synthetic image formed based upon the plural ultrasonic reception beams.

2. The ultrasonic diagnostic apparatus according to claim 1, wherein the beamformer is configured to:
form a first ultrasonic reception beam for an image of a biological tissue of a subject;
form a second ultrasonic reception beam for a biopsy needle inserted into the biological tissue; and
set a width of the second ultrasonic reception beam in the elevation direction such that a size of the second ultrasonic reception beam is capable of covering the biopsy needle outside of a range of the first ultrasonic reception beam.

3. The ultrasonic diagnostic apparatus according to claim 2, wherein the synthetic image is formed by synthesizing data based upon the first ultrasonic reception beam and data based upon the second ultrasonic reception beam.

4. The ultrasonic diagnostic apparatus according to claim 2, wherein the beamformer is configured to:
set a central frequency of a second ultrasonic transmission beam for acquiring the second ultrasonic reception beam lower than a central frequency of a first ultrasonic transmission beam for acquiring the first ultrasonic reception beam;
transmit the second ultrasonic transmission beam in a direction generally orthogonal to a planned insertion path of the biopsy needle; and
form the second ultrasonic reception beam in the direction generally orthogonal to the planned insertion path.

5. The ultrasonic diagnostic apparatus according to claim 3, wherein the beamformer is configured to:
set a central frequency of a second ultrasonic transmission beam for acquiring the second ultrasonic reception beam lower than a central frequency of a first ultrasonic transmission beam for acquiring the first ultrasonic reception beam;
transmit the second ultrasonic transmission beam in a direction generally orthogonal to a planned insertion path of the biopsy needle; and
form the second ultrasonic reception beam in the direction generally orthogonal to the planned insertion path.

6. The ultrasonic diagnostic apparatus according to claim 2, wherein the beamformer is configured to set a reception gain of the second ultrasonic reception beam higher in a first region in which the biopsy needle can be inserted than in a second region outside of the first region.

7. The ultrasonic diagnostic apparatus according to claim 3, wherein the beamformer is configured to set a reception gain of the second ultrasonic reception beam higher in a first region in which the biopsy needle can be inserted than in a second region outside of the first region.

8. The ultrasonic diagnostic apparatus according to claim 4, wherein the beamformer is configured to set a reception gain of the second ultrasonic reception beam higher in a first region in which the biopsy needle can be inserted than in a second region outside of the first region.

9. The ultrasonic diagnostic apparatus according to claim 5, wherein the beamformer is configured to set a reception gain of the second ultrasonic reception beam higher in a first region in which the biopsy needle can be inserted than in a second region outside of the first region.

10. The ultrasonic diagnostic apparatus according to claim 6, wherein the first region is set with the planned insertion path defined as a reference.

11. The ultrasonic diagnostic apparatus according to claim 7, wherein the first region is set with the planned insertion path defined as a reference.

12. The ultrasonic diagnostic apparatus according to claim 8, wherein the first region is set with the planned insertion path defined as a reference.

13. The ultrasonic diagnostic apparatus according to claim 9, wherein the first region is set with the planned insertion path defined as a reference.

14. The ultrasonic diagnostic apparatus according to claim 1, wherein the beamformer is configured to form the plural ultrasonic reception beams based upon the respective echo signals acquired by plural ultrasonic transmission beams, each ultrasonic transmission beam having a different width in the elevation direction.

15. The ultrasonic diagnostic apparatus according to claim 14, wherein the beamformer is configured to:
form a first ultrasonic transmission beam for an image of a biological tissue of a and subject;
form a second ultrasonic transmission beam for a biopsy needle inserted into the biological tissue; and
set a width of the second ultrasonic transmission beam in the elevation direction such that a size of the second ultrasonic transmission beam is capable of covering the biopsy needle outside of a range of the first ultrasonic transmission beam.

16. The ultrasonic diagnostic apparatus according to claim 1, wherein the beamformer is configured to form the plural ultrasonic reception beams based upon an echo signal acquired by one ultrasonic transmission beam for the one transmission/reception surface.

17. The ultrasonic diagnostic apparatus according to claim 2, wherein the beamformer is configured to set the width of the second ultrasonic reception beam a type of the biopsy needle, the type input on an operation unit.

18. A control program of an ultrasonic diagnostic apparatus, the control program configured to cause a computer to execute:
a beamforming function that forms an ultrasonic reception beam by performing delay addition to an echo signal received by each of a plurality of ultrasonic transducers, and that forms plural ultrasonic reception beams, each ultrasonic reception beam having a different width in an elevation direction, the plural ultrasonic reception beans formed for one transmission/reception surface by adjusting a delay time in the delay addition, and
a display control function that displays a synthetic image formed based upon the plural ultrasonic reception beams.

19. A method of operating an ultrasonic diagnostic apparatus that includes plural ultrasonic transducers arranged in an elevation direction, the method comprising:
forming, using a beamformer, a plurality of ultrasonic reception beams by performing delay addition to an echo signal received by each of the ultrasonic transducers, each ultrasonic reception beam having a different width in the elevation direction, wherein the plurality of ultrasonic reception beams are formed for one transmission/reception surface by adjusting a delay time in the delay addition; and
displaying, using a display control unit, a synthetic image that is formed based upon the plurality of ultrasonic reception beams.

20. The method according to claim 19, wherein forming a plurality of ultrasonic reception beams comprises:
forming a first ultrasonic reception beam for an image of a biological tissue of subject;
forming a second ultrasonic reception beam for a biopsy needle inserted into the biological tissue; and
setting a width of the second ultrasonic reception beam in the elevation direction such that a size of the second ultrasonic reception beam is capable of covering the biopsy needle outside of a range of the first ultrasonic reception beam.
